(19) United States
(12) Patent Application Publication
(10) Pub. No.: US 2024/0268782 A1
(43) Pub. Date: Aug. 15, 2024

(54) LUNG ABNORMALITIES DETECTION SYSTEM, A WEARABLE DEVICE, AND METHOD THEREFOR

(71) Applicant: MG Health Tech LLC, Allen, TX (US)

(72) Inventors: Manikandan Muthu, Salem (IN); Ganesh Natesan, Edaipadi (IN)

(21) Appl. No.: 18/648,371

(22) Filed: Apr. 27, 2024

(51) Int. Cl. A61B 7/00; A61B 5/00; A61B 5/08

(52) U.S. Cl. CPC A61B 7/003; A61B 5/0806; A61B 5/6805; A61B 2562/0285

(57) ABSTRACT

The present disclosure is directed towards a wearable device, a system and a method for detecting lung diseases that is non-invasive and leads to more accurate results. The wearable device (600) for detection of lung abnormalities includes a triboelectric/piezoelectric nanogenerator) sensor (604) and at least one acoustic sensor (602). The triboelectric/piezoelectric nanogenerator sensor is configured to be held on abdominal region of a user to continuously monitor the abdominal region's expansion and contraction (due to inhalation and exhalation) and generate a first electric signal accordingly, and the at least one acoustic sensor is configured to be held on an auscultation site of the user to continuously capture respiratory sounds from the user's body and generate a second electric signal accordingly. The first electric signal and the second electric signal are used to detect lung abnormalities of the user.

### TECHNICAL FIELD

The present disclosure is directed to detection of lung diseases. More particularly it is directed towards a wearable device, a system and a method for detecting lung diseases that is non-invasive and leads to more accurate results.

### BACKGROUND

Pulmonary function testing (PFT) is a complete evaluation of the respiratory system including patient history, physical examinations, and tests of pulmonary function. The primary purpose of pulmonary function testing is to identify the severity of pulmonary impairment. Pulmonary function testing has diagnostic and therapeutic roles and helps clinicians answer some general questions about patients with lung disease. PFTs are normally performed by a pulmonary function technician, respiratory therapist, respiratory physiologist, physiotherapist, pulmonologist, or general practitioner.

Several technologies exist for monitoring lung health and diagnosing diseases such as, pulmonary function tests (PFTs), arterial blood sampling, chest X-rays, tomographic scans, respiratory inductive plethysmography (RIP), and optoelectronic plethysmography.

These technologies use devices and methods that need human effort to evaluate lung function. Incorporating the human element may sometimes result in a lack of signal quality, and a lack of accuracy and reliability in predicting breathing patterns with consequent problems such as erroneous diagnosis.

Most of the traditional non-invasive methods require patients to be monitored in a clinical setting, disrupting their daily routine and failing to collect the data continuously. Moreover, these conventional devices are bulky and can easily slip, making them unsuitable for assessing patient's respiratory conditions throughout the day in their natural surroundings.

Further, conventional signal processing has shown poor signal waveform and undesirable respiratory signals, leading to incorrect predictions. Also, conventional devices operate based on the external battery or power module to power the sensor, which limits their device's mobility.

The conventional devices have additional drawbacks such as lack of signal quality, lack of accessible and affordable approaches for measuring respiratory rate, error in reading breathing pattern signals, noise level in the device data, inadequacy of conventional signal processing techniques and mechanical incompatibility with the human skin resulting in low stability and error in data acquisition. Further, the inadequacy of conventional signal processing techniques results in low accuracy in predicting human breathing patterns, and lack of noise level reduction in the device data.

There exists, therefore, a need for an improved system and method for the detection of anomalies in human respiration patterns that overcomes the above-mentioned challenges.

### SUMMARY

The present invention is directed to a system and a method for detecting lung diseases. More particularly it is directed towards such a system and method that is non-invasive and leads to more accurate results.

It is an object of the present disclosure to provide a system and a method for lung abnormalities detection that does not need human effort and is non-invasive.

It is another object of the present disclosure to provide a system and a method that provides for better signal quality and better analysis of breathing patterns and consequent diagnosis.

It is yet another object of the present disclosure to provide a system and a method that can work and collect relevant data continuously even in a non-clinical setting.

It is an object of the present disclosure to provide for a system and a method that uses light devices for its functioning.

It is another object of the present disclosure to provide for a system and a method that provides for an accessible and affordable approach for measuring respiratory rate, reduces error in reading breathing pattern signals, and reduces noise level in device data.

It is yet another object of the present disclosure to provide for a system and a method that is compatible with human skin thus providing high stability and accuracy in data prediction.

Accordingly, to overcome the above-mentioned challenges, the present disclosure provides a sensing device that may be a wearable triboelectric/piezoelectric nanogenerator sensing device or a system to monitor human respiratory patterns and a sliding mechanism of sensing device that works based on the changes in the circumference of the abdomen while breathing IN and OUT. The changes in the circumference of the abdomen while breathing plays a key role in signal generation and it occurs naturally without affecting daily activities.

The sensing device (interchangeably hereinafter referred to as “wearable device”) may include a versatile characteristic material for increasing durability and reducing the chances of performance losses. A water-resistant material may also be used for encapsulation to resist humidity influences. The triboelectric/piezoelectric nanogenerator may convert biomechanical energy to electric signal due to the inherent characteristics of triboelectric/piezoelectric, like inexpensive fabrication, high sensibility, flexibility, and excellent adaptability.

In the sliding mode, the triboelectric nanogenerator operates; fundamentally by tribo electrification effect and electrostatic induction. It works when two different polarized surface materials contact and separate each other. In human respiration, breathing results in the abdominal cavity regularly expanding and contracting as it inhales and exhales. In case of piezoelectric sensor, when mechanical stress or deformation occurs on the piezo material which results in the generation of electric charges within the material. Therefore, the variations in lower, middle, and upper abdomen circumference would be compatible with the sliding mode triboelectric/piezoelectric nanogenerator for monitoring human respiration. The triboelectric nanogenerator sensing device may also be implemented using other principles such as tribovoltaic, electrostatic induction, etc.

The triboelectric/piezoelectric nanogenerator sensing device construction consists of a top and bottom layer with the metal/carbon/polymer/cloth-based electrode and antistatic layer. The entire device is built on a flexible substrate with biocompatible and recyclable materials. The bioresorbable dynamic covalent polyurethane serves as an encapsulation base material. The polymer/semiconductor layers have been selected as active triboelectric/piezoelectric layer materials based on their surface charge density. The thickness of both triboelectric/piezoelectric nanogenerator layers will be optimized to obtain quality signal waveform and control the parasitic capacitance.

Each cyclic process consists of four operations, namely primary contact, inward sliding, dual time, and outward sliding, respectively. In direct contact (each overlapping); the positive and negative triboelectric material surfaces completely touch each other, which enable a static charge balance without any charge transfer to the external circuit. In the case of respiration inhalation, the tribo pair slides outward, causing the abdominal cavity to expand and generate an electric signal via electrostatic induction. On the other hand, reverse electrostatic induction occurs when exhalation causes the tribo layers to slide inward. Therefore, when the sensor deforms periodically caused by slide inward and outward, the electrons are driven between two electrodes back and forth via an external circuit with the alternating current signal. As a result, the sensing device may monitor the respiration patterns of the patient.

Any sweat-resistant materials, anti-allergic materials, shock absorbing material, quick release mechanisms may be used in strap. For patient or user comfort, flexibility, adaptability and secure fit to the body, the strap may use elastic-based, silicone-based, reflective, textile-based materials, or combination of these materials.

Any one of the encapsulation techniques such as epoxy resins, silicone sealants, polyurethane potting compounds, acrylic resin, parylene coating, fluorosilicone, polyethylene (PE), and polypropylene (PP) enclosure, conformal coatings, nanocoatings, glass or ceramic encapsulation, may be used to protect the sensors from dust, water, sweat or any unwanted thing that reduces the performance of the sensor, but this encapsulation does not affect the sensor's actual performance. Amphiphobic coating may also be used in encapsulation to resist both water and oil.

For the triboelectric/piezoelectric sensor to slide inward and outward as per the lower, middle, and upper abdomen movement structures like ball-bearing, spring-assisted, E-skin, silicon elastomer, self-healing, and elastic structures may be used.

To save energy, the faulty and uninterested readings will be eliminated and then transmitted to the detection unit. The faulty readings are well beyond the range of human capacity and potentially arise from a rare malfunction in the associated instrumentation system, and the uninteresting readings are those that show minimal deviation from the immediately preceding readings, and hence transmitting them to the detection unit will not provide any useful insight. A large percentage of readings are expected to be uninteresting, and eliminating these at the transmitting side itself will result in large savings in terms of energy. Assessment of faulty and uninteresting readings was done through the normal range of the human body and simple calculations involving computing the mean and variance of continuously monitored data.

The centralized server may be linked to the sensing devices through a network. The centralized server may be implemented using any or a combination of hardware-based, software-based, edge-based, network-based computing devices, or a cloud-based computing device.

The network may be a wireless network, a wired network, or a combination thereof that can be implemented as one of the different types of networks, such as Intranet, Local Area Network (LAN), Wide Area Network (WAN), Internet, Bluetooth, Wi-Fi (Wireless Fidelity), Cellular network, Zigbee, Satellite communication, Radio Frequency Identification, Long Range (LoRa) communication, and the like. The network either be a dedicated network or a shared network. The shared network can represent an association of different types of networks that can use a variety of protocols, for example, Hypertext Transfer Protocol (HTTP), Transmission Control Protocol/Internet Protocol (TCP/IP), Wireless Application Protocol (WAP), and the like.

The centralized server may include a detection unit. The detection unit may be present within the centralized server and may be in communication with the sensing device over the network. The detection unit may be present within the sensing device as well based on the scope of the invention.

The detection unit may be implemented as a processing resource and may be implemented as a combination of a transceiver and a processing resource. The detection unit may be capable of receiving data, processing it, and transmitting data.

In operation, the receiver unit will receive the signals from the sensing devices associated with the patient. Based on the received signals, the detection unit may extract the monitored respiration patterns of the patient.

The receiver unit may generate a report of a patient which includes the details of the analyzed breathing pattern. Such a report may be displayed on either the user device or display device and it may be transmitted over the network to the medical practitioner. So that the medical condition of the patient may be determined and the medicines may be instructed accordingly.

The receiver unit may be implemented with the computing device, but the computing device may be in another geographical location as that of the sensing device. In that case, the system may be implemented over a cloud network. For example, the patient may be wearing the sensing device at their residential facilities, and the medical practitioner at the hospital may be monitoring the patient.

The computing device may be operated either by the patient himself or by the medical practitioner. The computing device may be implemented such as a mobile phone, a personal computer, a laptop, a handheld device, a wearable computing device, a portable computer, a display device, etc., which includes a Graphical User Interface (GUI) that allows the operator to interact with it.

The processing engine(s) can be implemented as a combination of hardware and programmable instructions to attain one or more functionalities. The processing engine(s) may be implemented by electronic circuitry, which can include a detection unit and other unit(s). The other unit(s) can implement functionalities that supplement applications or functions performed by the computing device or the processing engine(s).

One or more processor(s) can be implemented as one or more microprocessors, microcomputers, microcontrollers, digital signal processors, central processing units, logic circuitries, and/or any devices that manipulate data based on operational instructions. Among other capabilities, one or more processor(s) are configured to fetch and execute computer-readable instructions stored in the memory of the computing device. The memory can store one or more computer-readable instructions or routines, which may be fetched and executed to create or share the data units over a network service. The memory can include any non-transitory storage device including, for example, volatile memory such as RAM, or non-volatile memory such as EPROM, flash memory, and the like.

The computing device can include the machine-readable storage medium storing the instructions and the processing resource to execute the instructions, or the machine-readable storage medium may be separate but accessible to the computing device and the processing resource. The database can include stored data or data generated as a result of functionalities implemented by any of the components of the processing engine(s).

The computing device may include a variety of interfaces to facilitate the communication of the computing device with various devices coupled to the computing device. The interface(s) may also provide a communication pathway for one or more components of the computing device.

The computing system includes an external storage device, one or more processors, a database, and one or more communication ports along with various modules associated with embodiments of the present invention. Examples of processors include, but are not limited to, an Intel® Itanium® or Itanium 2 processor(s), or AMD® Opteron® or Athlon MP® processor(s), Motorola® lines of processors, FortiSOC™ system on a chip processors or other future processors. Communication port can be any of an RS-232 port for use with a modem-based dialup connection, a 10/100 Ethernet port, a Gigabit or 10 Gigabit port using copper or fiber, a serial port, a parallel port, or other existing or future ports. The communication ports may be chosen depending on a network, such as a Local Area Network (LAN), Wide Area Network (WAN), or any network to which the computer system connects.

The Bus communicatively couples the processor(s) with the storage and communication blocks. The bus can be a Peripheral Component Interconnect (PCI)/PCI Extended (PCI X) bus, Small Computer System Interface (SCSI), USB, or the like for connecting the expansion cards, drives, and other subsystems as well as other buses, such as front side bus (FSB), which connects the processor to the software system. To support direct operator interaction with the invention's computing system, interfaces such as display, keyboard, and cursor control devices may also be coupled to a bus or connected through a communication port. External storage devices can be any kind of external hard drives, floppy drives, IOMEGA® Zip Drives, Compact Disc-Read Only Memory (CD-ROM), Compact Disc-Re-Writable (CD-RW), Digital Video Disk-Read Only Memory (DVD-ROM). Components described above are meant only to show the various possibilities and these cannot limit the scope of the present disclosure.

For storing static information like start-up or BIOS instructions for the processor, Read Only Memory (ROM) may be used. The Read Only Memory (ROM) includes any static storage device(s) but is not limited to, Programmable Read Only Memory (PROM) chips for storing static information. The main memory can be Random Access Memory (RAM) or any other dynamic storage. Mass storage solutions devices may also be used to store information and instructions, which include, but are not limited to, Parallel Advanced Technology Attachment (PATA) or Serial Advanced Technology Attachment (SATA) hard disk drives or solid-state drives (internal or external like Universal Serial Bus (USB) and/or Firewire interfaces, one or more optical discs, Redundant Array of Independent Disks (RAID) storage.

### BRIEF DESCRIPTION OF THE DRAWINGS

The diagrams are for illustration only, which thus is not a limitation of the present disclosure, and wherein:

FIG. 1 illustrates a block diagram of the system disclosed in accordance with an exemplary embodiment of the present disclosure.

FIG. 2 illustrates lung auscultation sites-anterior and posterior view in accordance with an exemplary embodiment of the present disclosure.

FIG. 3 illustrates by means of a flow chart the method of working of the proposed system in accordance with an exemplary embodiment of the present disclosure.

FIG. 4 illustrates the shape of an acoustic sensor patch in accordance with an exemplary embodiment of the present disclosure.

FIG. 5 illustrates a sensing device in accordance with an exemplary embodiment of the present disclosure.

FIG. 6 illustrates the anterior and posterior view of the human body after the sensor placement in accordance with an exemplary embodiment of the present disclosure.

FIG. 7 illustrates auscultation sites in accordance with an exemplary embodiment of the present disclosure.

FIG. 8 illustrates working of the proposed system in accordance with an exemplary embodiment of the present disclosure.

FIG. 9 illustrates a method for lung abnormalities detection in accordance with an exemplary embodiment of the present disclosure.

### DETAILED DESCRIPTION

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

Each of the appended claims defines a separate invention, which for infringement purposes is recognized as including equivalents to the various elements or limitations specified in the claims. Depending on the context, all references below to the “invention” may in some cases refer to certain specific embodiments only. In other cases it will be recognized that references to the “invention” will refer to subject matter recited in one or more, but not necessarily all, of the claims.

Various terms as used herein are defined below. To the extent a term used in a claim is not defined below, it should be given the broadest definition persons in the pertinent art have given that term as reflected in printed publications and issued patents at the time of filing.

In the following description, numerous specific details are set forth in order to provide a thorough understanding of embodiments of the present invention. It will be apparent to one skilled in the art that embodiments of the present invention may be practiced without some of these specific details.

Embodiments of the present invention include various steps, which will be described below. The steps may be performed by hardware components or may be embodied in machine-executable instructions, which may be used to cause a general-purpose or special-purpose processor programmed with the instructions to perform the steps. Alternatively, steps may be performed by a combination of hardware, software, and firmware and/or by human operators.

Embodiments of the present invention may be provided as a computer program product, which may include a machine-readable storage medium tangibly embodying thereon instructions, which may be used to program a computer (or other electronic devices) to perform a process. The machine-readable medium may include, but is not limited to, fixed (hard) drives, magnetic tape, floppy diskettes, optical disks, compact disc read-only memories (CD-ROMs), and magneto-optical disks, semiconductor memories, such as ROMs, PROMs, random access memories (RAMs), programmable read-only memories (PROMs), erasable PROMs (EPROMs), electrically erasable PROMs (EEPROMs), flash memory, magnetic or optical cards, or other type of media/machine-readable medium suitable for storing electronic instructions (e.g., computer programming code, such as software or firmware).

Accordingly, embodiments of the present invention relates to the usage of acoustic body sensors along with Triboelectric or piezoelectric Nanogenerator, with a specific focus on the application of wearable sensors for measuring signals originating from the body for detecting the lung diseases.

The acoustic sensor captures respiratory sounds from the body, which is just a mechanical vibration generated by the respiration that is translated into electrical signals. Capturing the movements continuously results in an Alternating Current (AC) or Direct current signal that reflects dynamic changes in voltage over time.

These captured signals are amplified and filtered with the help of signal conditioning circuits and further the conditioned analog signal is converted to digital format. The features of the digital signals are extracted using machine learning algorithms and sound classifying algorithm is applied to those features for diagnosing lung diseases.

In an embodiment, the present disclosure elaborates upon a system for lung abnormalities detection. The system can include a triboelectric/piezoelectric nanogenerator sensor and at least one acoustic sensor wherein the triboelectric/piezoelectric nanogenerator sensor can be configured to be held on abdominal region of a user to continuously monitor the abdominal region's expansion and contraction (due to inhalation and exhalation) and can generate a first electric signal accordingly, and the at least one acoustic sensor can be configured to be held on an auscultation site of the user to continuously capture respiratory sounds from the user's body and can generate a second electric signal accordingly, and wherein the first electric signal and the second electric signal can be used to detect lung abnormalities of the user.

In an exemplary embodiment, the TENG or a piezo sensor and the at least one acoustic sensor can be both configured on one wearable device worn by the user.

In an exemplary embodiment, the first electric signal and the second electric signal can undergo signal conditioning, analog to digital conversion, sound classification and feature extraction to generate data usable to detect the lung abnormalities.

In an exemplary embodiment, the first electric signal and the second electric signal can be used to train machine learning algorithms to enable accurate differentiation of diverse human breathing sounds.

In an exemplary embodiment, the sound classification can use reliable sound classification algorithms developed to classify respiratory sounds of the user.

In an exemplary embodiment, the feature extraction can use a suitable feature extraction algorithm to extract amplitude, frequency, and time from any or a combination of the first electric signal and the second electric signal.

In an exemplary embodiment, in case of detection of severe abnormality in lung function of a user, the system can generate an alarm signal and can use Global Positioning System to track the user's accurate location for provisioning of emergency services to the user and information to emergency contacts of the user.

In an exemplary embodiment, the present disclosure elaborates upon a method for lung abnormalities detection.

The integration of acoustic sensors into wearables, such as smart garments or patches, exemplifies a convergence of sensor design, including miniaturization and enhanced sensitivity, have played a pivotal role in making these wearable devices feasible and effective.

As industry trends converge, wearable sensors hold the potential to redefine healthcare practices by enabling early disease detection, continuous health tracking, and personalized patient care.

In another embodiment, the present disclosure elaborates upon an intelligent sensing device for respiratory monitoring and a method for detecting lung abnormalities by the analysis of the vital signs of humans. It is an innovative technology for detecting respiratory patterns that uses a wearable sensor system for human respiratory monitoring.

In an exemplary embodiment, the thickness of both tribo/piezo layers may be optimized to obtain quality signal waveform and control the parasitic capacitance.

In an exemplary embodiment, each cyclic process of the triboelectric nanogenerator consists of four operations, namely primary contact, inward sliding, dual time, and outward sliding, respectively. In direct contact (each overlapping), the positive and negative triboelectric material surfaces completely touch each other, which enables a static charge balance without any charge transfer to the external circuit.

In an exemplary embodiment, at time of respiration inhalation, the abdominal cavity expands causing the sensor slides outward. This causes and generates an electric signal.

In an exemplary embodiment, at time of exhalation the abdominal cavity contracts causing the sensor slides inward. This cause reverse.

In this manner, the tribo/piezo sensor deforms during inhalation and exhalation due to inward and outward sliding movement. Thereby the electrons are driven between two electrodes back and forth via an external circuit with the alternating current signal. As a result, the sensing device may monitor the respiration patterns of the patient.

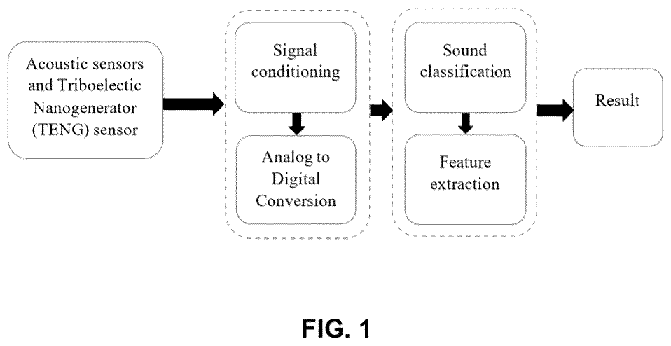

The present disclosure concerns acoustic body sensors along with triboelectric or piezoelectric nanogenerator, with a specific focus on the application of wearable sensors for measuring signals originating from the body for detecting the lung diseases. FIG. 1 illustrates a block diagram of the system disclosed in accordance with an exemplary embodiment of the present disclosure.

As shown in FIG. 1, the operational mechanism of the sensing device in human respiration, breathing results in the abdominal cavity regularly expanding and contracting as it inhales and exhales. Therefore, the variations in abdomen circumference would be compatible with the sliding mode triboelectric/piezoelectric nanogenerator for monitoring human respiration.

The acoustic sensor captures respiratory sounds from the body, which is just a mechanical vibration generated by the respiration that are translated into electrical signals. Capturing the movements continuously resulting in an Alternating Current (AC) signal that reflects dynamic changes in voltage over time.

The present disclosure uses a sensing module consists of Triboelectric/piezoelectric Nanogenerator sensor for detecting small movement occurs in chest or abdominal region and one or more acoustic sensors for detecting minute pressure fluctuation or mechanical vibration that occurs due to respiratory activity of a human. The acoustic sensors are placed on some auscultation sites of human body, so that the shape of wearable is to be designed based on the chosen auscultation sites.

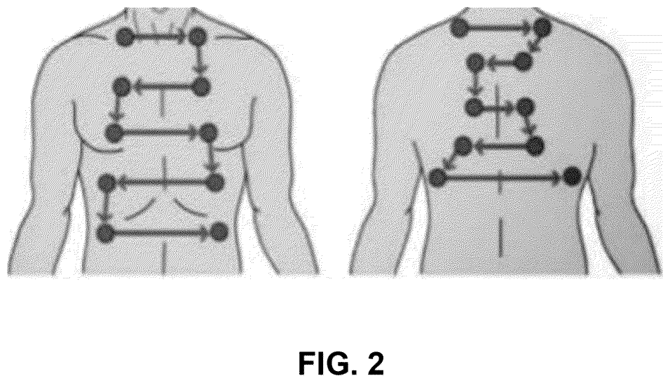

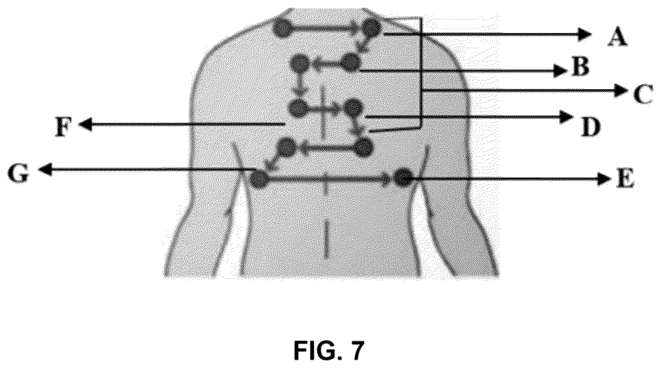

FIG. 2 illustrates lung auscultation sites-anterior and posterior view in accordance with an exemplary embodiment of the present disclosure. In an exemplary embodiment, lung auscultation sites can be those sites from which the pressure fluctuation or mechanical vibration of lungs and/or heart can be heard. In an example, the auscultation sites can be any or all of the sites as elaborated in FIG. 7 below.

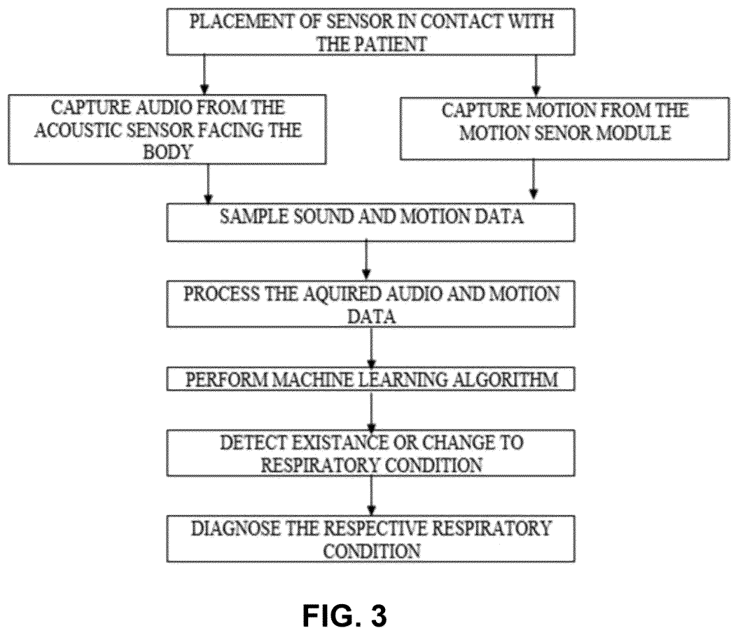

FIG. 3 illustrates by means of a flow chart the method of working of the proposed system in accordance with an exemplary embodiment of the present disclosure.

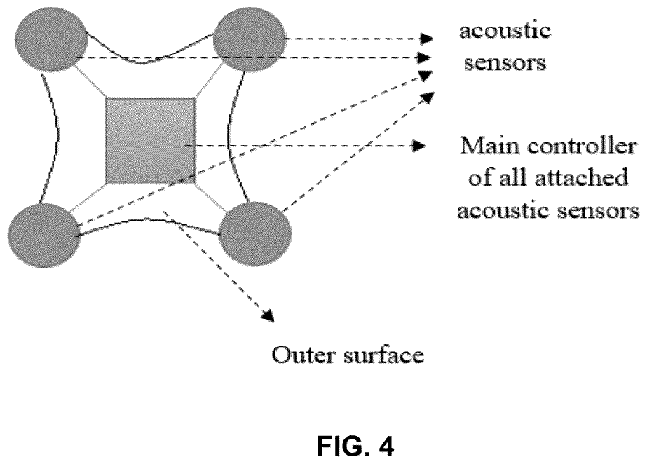

FIG. 4 illustrates the shape of an acoustic sensor vest in accordance with an exemplary embodiment of the present disclosure.

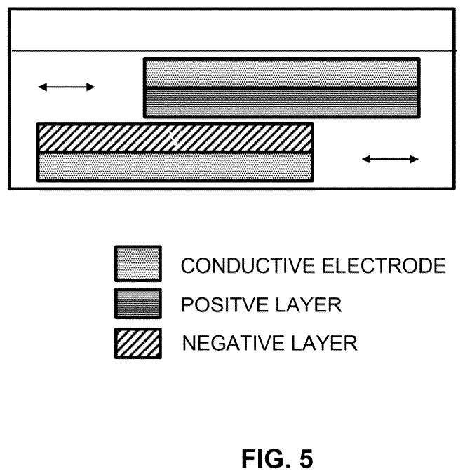

FIG. 5 illustrates a Triboelectric Nanogenerator sensing device in accordance with an exemplary embodiment of the present disclosure. As shown in FIG. 5, the sensing device may be a wearable triboelectric nanogenerator sensing device to monitor human respiratory patterns and the sliding mechanism of the triboelectric nanogenerator sensing device works based on the changes in the circumference of the abdomen while breathing IN and OUT. Which plays a key role in signal generation and it occurs naturally without affecting daily activities.

The sensing device may include a versatile characteristic material for increasing durability and reducing the chances of performance losses. A water-resistant material may also be used for encapsulation to resist humidity influences. The triboelectric/piezoelectric nanogenerator may convert biomechanical energy to electric signal due to the inherent characteristics of triboelectric/piezoelectric nanogenerator, like inexpensive fabrication, high sensibility, flexibility, and excellent adaptability.

The triboelectric/piezoelectric nanogenerator sensing device construction consists of a top and bottom layer with the metal/carbon/polymer/cloth-based electrode. The entire device is built on a flexible substrate with biocompatible and recyclable materials. The bioresorbable dynamic covalent polyurethane serves as an encapsulation base material. The polymer/semiconductor layers have been selected as active triboelectric/piezoelectric layer materials based on their surface charge density. The thickness of both triboelectric/piezoelectric nanogenerator layers will be optimized to obtain quality signal waveform and control the parasitic capacitance.

Each cyclic process consists of four operations, namely primary contact, inward sliding, dual time, and outward sliding, respectively. In direct contact (each overlapping), the positive and negative triboelectric material surfaces completely touch each other, which enables a static charge balance without any charge transfer to the external circuit. In the case of respiration inhalation, the tribo pair slides outward, causing the abdominal cavity to expand and generate an electric signal via electrostatic induction. On the other hand, reverse electrostatic induction occurs when exhalation causes the tribo layers to slide inward. Therefore, when the tribo layers periodically slide inward and outward, the electrons are driven between two electrodes back and forth via an external circuit with the alternating current signal. As a result, the sensing device may monitor the respiration patterns of the patient.

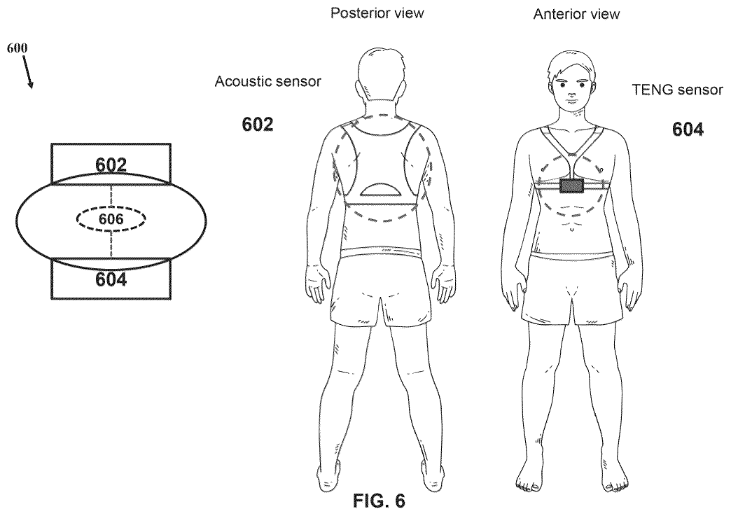

FIG. 6 illustrates anterior and posterior view of the human body after the sensor placement in accordance with an exemplary embodiment of the present disclosure.

In a preferred embodiment, a wearable device (600) for detection of lung abnormalities is disclosed. The wearable device (600) includes one or more acoustic sensors (602) adapted to be positioned at one or more auscultation sites a human body, one or more triboelectric/piezoelectric nanogenerator sensors (604) operatively coupled with the one or more acoustic sensors and are adapted to be positioned on an abdomen region of the human body, and a controller (606).

The one or more acoustic sensors are configured to detect mechanical vibration at the one or more auscultation sites to generate a first signal.

The one or more triboelectric/piezoelectric nanogenerator sensors are configured to capture biomechanical energy generated by changes in circumference of the abdomen while breathing activity to thereby convert the captured biomechanical energy into a second electric signal.

The controller (606) is configured to retrieve the first electric signal and the second electric signal and process the retrieved first electric signal and the retrieved second electric signal for detection of the lung abnormalities.

In an implementation of this embodiment, one or more auscultation sites are present on the chest and the abdominal part of the human body.

In an implementation of this embodiment, the circumference of the abdomen is associated with an abdomen circumference.

In an implementation of this embodiment, the mechanical vibration is associated with minute pressure fluctuations in a chest region that occurs during respiratory activity, and wherein the changes in circumference of the abdomen are associated with a movement in the chest region that occurs during respiratory activity.

In an implementation of this embodiment, the wearable device is any or a combination of a garment, a smart apparel, a smart band, a strap, a patch, a smartwatch, a wristband, and a smart jewelry.

In an implementation of this embodiment, the one or more triboelectric nanogenerator sensors are configured to operate in a sliding mode to generate a triboelectrification effect with electrostatic induction, such that the changes in circumference of the abdomen causes two different polarized surface materials of the triboelectric to contact and separate from each other.

In an implementation of this embodiment, the one or more triboelectric/piezoelectric nanogenerator sensors are configured to operate in a cyclic process that consists of one or more operations selected from any or a combination of a primary contact, an inward sliding, and an outward sliding.

In the primary contact, a positive and a negative triboelectric/piezoelectric material surfaces touch (overlap) each other such that the touch (overlap) enables a static charge balance without any charge transfer to an external circuit.

In the inward sliding and the outward sliding, during respiration inhalation, the positive and the negative triboelectric material surfaces slides outward, causing an abdominal cavity to expand and generate an electric signal via electrostatic induction, and wherein during reverse electrostatic induction occurs, the exhalation causes the positive and the negative triboelectric/piezoelectric material surfaces to slide inward, and therefore, when the positive and the negative triboelectric material surfaces periodically slide inward and outward, electrons are driven between two electrodes back and forth via the external circuit with the alternating current signal

In an implementation of this embodiment, the controller comprises an artificial intelligence (AI) technique or a machine learning technique to process the retrieved first electric signal and the retrieved second electric signal for detection of the lung abnormalities.

In an exemplary implementation, the AI/ML techniques operate in following manner:

Data Collection: High-quality lung sound recordings (the retrieved first electric signal and the retrieved second electric signal) are collected using specialized equipment such as electronic stethoscopes or digital recording devices.

Data Preprocessing: The collected data may undergo preprocessing steps such as noise reduction, filtering, and segmentation to extract relevant features.

Feature Extraction: Features are extracted from the preprocessed lung sound data. These features may include time-domain features, frequency-domain features, or other signal characteristics that are indicative of abnormalities.

Labeling: Each sample in the dataset is labeled with the corresponding lung condition or abnormality, such as pneumonia, wheezing, crackles, or normal.

Model Training: Supervised learning algorithms such as Support Vector Machines (SVM), Random Forests, Convolutional Neural Networks (CNNs), or Recurrent Neural Networks (RNNs) are trained on the labeled dataset. The algorithm learns to map the extracted features to the corresponding lung conditions.

Model Evaluation: The trained model is evaluated using separate testing data to assess its performance in accurately detecting lung abnormalities. Evaluation metrics such as accuracy, sensitivity, specificity, and area under the receiver operating characteristic curve (AUC-ROC) are commonly used.

Deployment and Integration: Once the model demonstrates satisfactory performance, it can be deployed in clinical settings for real-time detection of lung abnormalities. Integration with existing healthcare systems or medical devices may be necessary for practical application.

Continuous Improvement: The model may undergo continuous refinement and improvement based on feedback from healthcare professionals and additional data collection to enhance its accuracy and reliability.

In another embodiment of the present disclosure, a system for detection of lung abnormalities is disclosed. The system includes one or more acoustic sensors (602), one or more triboelectric/piezoelectric nanogenerator sensors (604), and a controller (606).

The one or more acoustic sensors (602) is adapted to be positioned at one or more auscultation sites a human body, the one or more acoustic sensors are configured to detect mechanical vibration at the one or more auscultation sites to generate a first signal.

The one or more triboelectric/piezoelectric nanogenerator sensors (604) is operatively coupled with the one or more acoustic sensors. The one or more triboelectric/piezoelectric nanogenerator sensors are adapted to be positioned on an abdomen region of the human body. The one or more triboelectric/piezoelectric nanogenerator sensors are configured to capture biomechanical energy generated by changes in circumference of the abdomen while breathing activity to thereby convert the captured biomechanical energy into a second electric signal.

The controller (606) is communicably coupled to the one or more acoustic sensors and the triboelectric/piezoelectric nanogenerator sensors. The controller is configured to retrieve the first electric signal and the second electric signal and process the retrieved first electric signal and the retrieved second electric signal for detection of the lung abnormalities. The controller is embedded in a computer system located at a remote location.

FIG. 7 illustrates auscultation sites as shown in FIG. 2. Following are exemplary sites:

A. Bronchial which is a Loud, High-pitched, Expiratory phase that is longer than the inspiratory phase. There is a gap between the inspiratory and the expiratory phase Pneumonia, dense lung fibrosis, lung abscess, or lung collapse associated with pleural effusion.

B. Bronchovesicular breath sounds (manubrium) which is an intermediate to bronchial and vesicular. inspiratory phase=expiratory phase.

C. Vesicular sound which is a soft, low-pitched, inspiratory phase (much longer) than the expiratory phase (passive process) sound produced because of the turbulence. There is no gap between the inspiratory and expiratory phases.

D. Crackles which is a Non-musical sound, Non-continuous sound-(Fine and coarse).

E. Wheeze which is a musical, high pitch, continuous, expiratory phase>inspiratory phase (can be heard in this phase also) due to a narrowed lumen because of constriction in the airway Polyphonic Wheeze (asthma and chronic and COPD)

F. Rhonchi which is a low pitch, continuous, bubbling, or rattling.

G. Stridor which is a Musical, Loud, Crowing, Whistling, High pitched, Inspiratory phase-extrathoracic obstruction (extrathoracic lesion) e.g. —laryngomalacia or vocal cord lesions, expiratory phase-intrathoracic obstruction e.g. —bonchomalacia, Tracheomalacia and external compression, Both phases-Fixed obstruction-Stenosis.

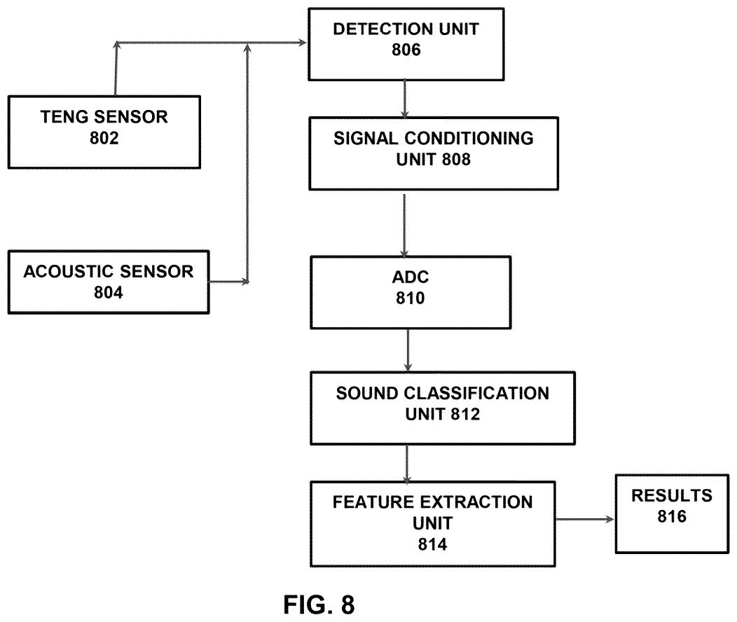

FIG. 8 illustrates working of the proposed system in accordance with an exemplary embodiment of the present disclosure.

In an aspect, the proposed system can have a triboelectric/piezoelectric nanogenerator sensor shown as 802 and at least one acoustic sensor shown as 804.

In another aspect, sensor 802 can be configured to be held on the abdominal region of a user (patient) so as to monitor abdominal region expansion and contraction (due to inhalation and exhalation of breath) continuously and generate electric signals accordingly.

In yet another aspect, sensor 804 can be configured to be held on an auscultation site of the patient so as to continuously capture respiratory sounds from the body and generate electric signals accordingly.

In an aspect, the electric signals generated by sensors 802 and 804 can be passed on by the proposed system to a detection unit shown as 806. Unit 806 device can be configured in one of the sensor bodies itself, or it can be part of a separate computing and processing resource having further units as shown in FIG. 8. Any of these units can be configured in the cloud using means known in the art and so can be remote from the user.

In an exemplary embodiment, it is possible that sensors 802 and 804 communicate with the detection unit 806 by means of Bluetooth or any similar near field communication technique. Unit 806 can communicate via Internet to other units further elaborated and the result can be printed out in a doctor's office remote from the user. Or sensors 802 and 804 can themselves be Internet enabled (by being configured as Internet of Things-IoT devices, for instance), can store their signals till Internet is available to them and then pass on their signals/data to unit 806. Thus, a user wearing sensors 802 and 804 can move anywhere and not be restricted to a clinical environment. All such embodiments are fully a part of the present disclosure.

In an aspect the electric signals generated by sensors 802 and 804 can be used to detect lung abnormalities of a user as further elaborated.

In an aspect disclosed system can further include signal conditioning unit 808, analog to digital converter (ADC) 810, sound classification unit 812, feature extraction unit 814 and results unit 816. Signal conditioning unit 808 can remove any noise from electrical signals being received from detection unit 806 and provide the cleaned signals to ADC 810. ADC 810 can receive the cleaned signals being received from unit 808 and convert them to digital signals which it can then provide to sound classification unit 812.

In another aspect, sound classification unit 812 can classify the digital signals received into various types of sounds and provide such sounds to feature extraction unit 814. Sound classification unit 812 can use sound classification algorithms to categorize/classify the respiratory sounds of the user.

In another aspect, feature extraction unit 814 can receive classified sounds from sound classification unit 812 and can extract relevant features such as amplitude, frequency, and time from such digital signals. Feature extraction unit can use suitable feature extraction algorithms for this purpose.

Such extracted features can be provided to a result unit shown as 816 that can, for instance, print them out. Further inspection of the printout of these extracted features can enable expert medical professionals (a lung disease doctor, for example) to make a proper diagnosis of lung disease, if any, being suffered by the user.

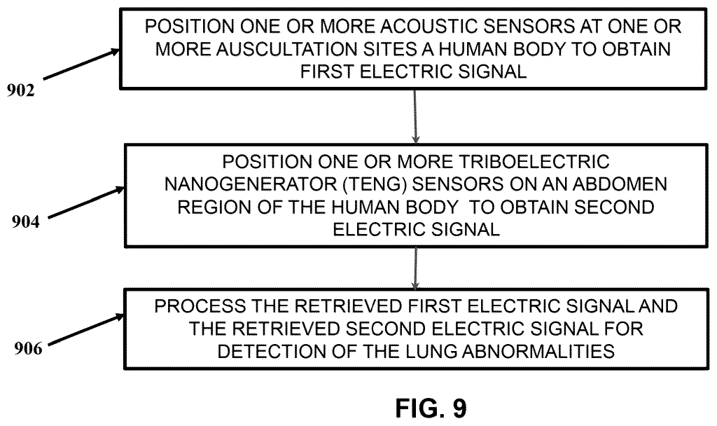

FIG. 9 illustrates a method for lung abnormalities detection in accordance with an exemplary embodiment of the present disclosure.

The method can also comprise the following steps.

At step 902, one or more acoustic sensors are positioned at one or more auscultation sites a human body. The one or more acoustic sensors are configured to detect mechanical vibration at the one or more auscultation sites to generate a first signal.

At step 904, one or more triboelectric/piezoelectric nanogenerator sensors are positioned on an abdomen region of the human body. The one or more triboelectric/piezoelectric nanogenerator sensors are configured to capture biomechanical energy generated by changes in circumference of the abdomen while breathing activity to thereby convert the captured biomechanical energy into a second electric signal.

At step 906, a controller retrieves the first electric signal and the second electric signal. Then them controller processes the retrieved first electric signal and the retrieved second electric signal for detection of the lung abnormalities.

At step 1, configuring a triboelectric/piezoelectric nanogenerator sensor to be held on abdominal region of a user to continuously monitor the abdomen region's expansion and contraction (due to inhalation and exhalation) and generating a first electric signal accordingly.

At step 2, configuring at least one acoustic sensor to be held on an auscultation site of the user to continuously capture respiratory sounds from the user's body and generate a second electric signal accordingly.

At step 3, using the first electric signal and the second electric signal to detect lung abnormalities of the user.

In this manner the proposed system uses a combination of TENG and one or more acoustic sensors attached to single wearable device for accuracy of diagnosing the lung diseases.

The proposed sensing device is a wearable triboelectric/piezoelectric nanogenerator along with acoustic sensors-based sensing device to monitor human respiratory patterns.

The triboelectric/piezoelectric nanogenerator sensor will be an alternative for supplying high-resolution signal waveform at low to high-frequency situations because of the changeable capacitance characteristic. The sliding triboelectric/piezoelectric nanogenerator mechanism is compatible with human breathing since the abdomens regularly expand and contract cyclically. Therefore, changing the abdominal circumference variation is a key element for electrical signal generation and is a behavior that occurs naturally without impacting people's daily activities.

The acoustic sensor captures respiratory sounds from the body, which is just a mechanical vibration generated by the respiration that are translated into electrical signals. Capturing the movements continuously resulting in an Alternating Current (AC) signal that reflects dynamic changes in voltage over time.

One or more acoustic sensors can be placed over various nodes, which are the auscultation sites of the lungs for accurate detection of normal and abnormal lung sounds like rhonchi, wheeze, crackles, stridor, pleural rub, etc.

The proposed approaches may be able to detect different human respiratory patterns such as eupnea, biot, bradypnea, sighing, tachypnea, Cheyne-stokes, and Kussmaul.

Machine learning algorithms can undergo training with obtained electrophysiological signals, enabling accurate differentiation of diverse human breathing sounds. Suitable feature extraction algorithms can be used to extract amplitude, frequency and time from the digital signal obtained.

In the case of severe abnormality in lung function, the sensor system may generate an alarm signal and may use Global Positioning System to track the patient's accurate location information for emergency services or designated contacts, aids a timely and effective response. As would be appreciated, such alarm signals and GPS tracking may be beneficial in a patient's critical condition.

In an aspect system disclosed can have the following operating ranges and devices there in can be made of different materials as described herein. Sensor Materials: Polymers/semiconductors.

In another aspect the electrode material for the triboelectric/piezoelectric nanogenerator sensor can be metal/carbon-based electrodes/cloth-based electrodes. The triboelectric/piezoelectric nanogenerator sensor can have an operational frequency of 0.05 Hz to 20 Hz. with a sensor material thickness of 0.001 mm to 5 mm and the electrode material thickness 0.01 μm to 1 mm. The dimensions of the triboelectric/piezoelectric nanogenerator sensor can be (L*W*T): (2-7 cm*2-7 cm*2-4 cm) but it can vary. The triboelectric/piezoelectric nanogenerator sensor may be configured in a belt strap made of polymer and cotton strap while the belt circumference can be 12 inches to 70 inches.

In yet another aspect the operational frequency for the acoustic sensors can be 20 Hz to 2000 Hz will vary and they may operate within a temperature range of minus 40 degrees Centigrade to 85 degrees Centigrade. The acoustic sensors can monitor a sound pressure level (SPL) of 20 to 200 pascal (30 to 60 dB SPL).

In another aspect the proposed system can be configured to monitor the following ranges of signals.
Respiratory Rate range: 4-70 bpm (Breaths per minute) (for adults); 4-120 bpm (for neonates)
Normal respiratory sound: 150-1000 Hz
Wheezing sound: >200 Hz (>250 ms)
Rhonchi sound: <200 Hz (>250 ms)
Coarse crackles: 200-2000 Hz (<30 ms)
Fine crackles: 200-2000 Hz (<10 ms)
Normal Inspiration time: 0.5 to 2 seconds
Normal Expiration time: 2 to 3.5 seconds
Tidal Volume: 300-500 ml (6-8 ml/kg)
Residual Volume: 1200 ml (20-25 ml/kg)
Expiratory Reserve Volume: 700-1200 ml
Inspiratory Reserve Volume: 1900-3300 ml
Functional Residual Capacity: 1800-2200 ml
Total Lung Capacity: 4-6 Liters

### Advantages of the Invention

The present disclosure provides for a system for lung abnormalities detection that does not need human effort and is non-invasive.

The present disclosure provides for a system that provides for better signal quality and better analysis of breathing patterns and consequent diagnosis.

The present disclosure provides for a system that can work and collect relevant data continuously even in a non-clinical setting.

The present disclosure provides for a system that uses light devices for its functioning.

The present disclosure provides for a system that provides for an accessible and affordable approach for measuring respiratory rate, reduces error in reading breathing pattern signals, and reduces noise level in device data.

The present disclosure provides for a system that is compatible with human skin thus providing high stability and accuracy in data prediction.

The present disclosure provides for a system that uses sensors that do not need external power or battery modules for their power, thus leading to high mobility.

We claim:

1. A wearable device (600) for detection of lung abnormalities, the wearable device comprising:
one or more acoustic sensors (602) adapted to be positioned at one or more auscultation sites a human body, the one or more acoustic sensors are configured to detect mechanical vibration at the one or more auscultation sites to generate a first signal;
one or more triboelectric/piezoelectric nanogenerator sensors (604) operatively coupled with the one or more acoustic sensors, the one or more triboelectric/piezoelectric nanogenerator sensors are adapted to be positioned on an abdomen region of the human body, and the one or more triboelectric/piezoelectric nanogenerator sensors are configured to capture biomechanical energy generated by changes in circumference of the abdomen while breathing activity to thereby convert the captured biomechanical energy into a second electric signal; and
a controller (606) configured to retrieve the first electric signal and the second electric signal and process the retrieved first electric signal and the retrieved second electric signal for detection of the lung abnormalities.

2. The wearable device as claimed in claim 1, wherein the one or more auscultation sites are present on a chest and an abdominal region part of the human body.

3. The wearable device as claimed in claim 1, wherein the circumference of the abdomen is associated with an abdomen circumference.

4. The wearable device as claimed in claim 1, wherein the mechanical vibration is associated with minute pressure fluctuations in a chest region that occurs during respiratory activity, and wherein the changes in circumference of the abdomen are associated with a movement in the chest region that occurs during respiratory activity.

5. The wearable device as claimed in claim 1, wherein the wearable device is any or a combination of a garment, a smart apparel, a smart band, a strap, a patch, a smartwatch, a wristband, and a smart jewelry.

6. The wearable device as claimed in claim 1, wherein the one or more triboelectric/piezoelectric nanogenerator sensors are configured to operate in a sliding mode to generate a triboelectrification/piezoelectric effect, such that:
for triboelectric sensor, the changes in circumference of the abdomen causes two different polarized surface materials of the triboelectric nanogenerator to contact and separate from each other; and
for piezoelectric sensor, when mechanical stress or sliding deformation occurs on the piezo material, electric charges are generated within the material.

7. The wearable device as claimed in claim 1, wherein the one or more triboelectric/piezoelectric nanogenerator sensors are configured to operate in a cyclic process that consists of one or more operations selected from any or a combination of a primary contact, an inward sliding, and an outward sliding, wherein:
in the primary contact, a positive and a negative triboelectric material surfaces touch (overlap) each other such that the touch (overlap) enables a static charge balance without any charge transfer to an external circuit;
in the inward sliding and the outward sliding, during respiration inhalation, the positive and the negative triboelectric material surfaces slides outward, causing an abdominal cavity to expand and generate an electric signal via electrostatic induction, and wherein during reverse electrostatic induction occurs, the exhalation causes the positive and the negative triboelectric material surfaces to slide inward, and therefore, when the positive and the negative triboelectric/piezoelectric material surfaces periodically slide inward and outward, electrons are driven between two electrodes back and forth via the external circuit with the alternating current signal.

8. The wearable device as claimed in claim 1, wherein the controller comprises an artificial intelligence (AI) technique or a machine learning technique to process retrieved first electric signal and the retrieved second electric signal for detection of the lung abnormalities.

9. The wearable device as claimed in claim 1, wherein the controller is configured to detect different human respiratory patterns such as eupnea, biot, bradypnea, sighing, tachypnea, Cheyne-stokes, and Kussmaul from first electric signal obtained using tribo-electric/piezo-electric nanogenerator.

10. The wearable device as claimed in claim 1, wherein the controller is configured to detect the normal and abnormal lung sounds like rhonchi, wheeze, crackles, stridor, and pleural rub, from second electric signal with the help of one or more acoustic sensors.

11. A system for detection of lung abnormalities, the system comprising:
one or more acoustic sensors (602) adapted to be positioned at one or more auscultation sites a human body, the one or more acoustic sensors are configured to detect mechanical vibration at the one or more auscultation sites to generate a first signal;
one or more triboelectric/piezoelectric nanogenerator sensors (604) operatively coupled with the one or more acoustic sensors, the one or more triboelectric/piezoelectric nanogenerator sensors are adapted to be positioned on an abdomen region of the human body, and the one or more triboelectric/piezoelectric nanogenerator sensors are configured to capture biomechanical energy generated by changes in circumference of the abdomen while breathing activity to thereby convert the captured biomechanical energy into a second electric signal; and
a controller (606) communicably coupled to the one or more acoustic sensors and the triboelectric/piezoelectric nanogenerator sensors, the controller configured to retrieve the first electric signal and the second electric signal and process the retrieved first electric signal and the retrieved second electric signal for detection of the lung abnormalities, and wherein the controller is embedded in a computer system located at a remote location.

12. A method for detection of lung abnormalities, the method comprising:
positioning (902) one or more acoustic sensors at one or more auscultation sites a human body, the one or more acoustic sensors are configured to detect mechanical vibration at the one or more auscultation sites to generate a first signal;
positioning (904) one or more triboelectric/piezoelectric nanogenerator sensors on an abdomen region of the human body, and the one or more triboelectric/piezoelectric nanogenerator sensors are configured to capture biomechanical energy generated by changes in circumference of the abdomen while breathing activity to thereby convert the captured biomechanical energy into a second electric signal; and
retrieving, by a controller, the first electric signal and the second electric signal;
processing (906), by a controller, the retrieved first electric signal and the retrieved second electric signal for detection of the lung abnormalities.
